(19) United States
(12) Patent Application Publication
(10) Pub. No.: US 2013/0035597 A1
(43) Pub. Date: Feb. 7, 2013

(54) ANORECTAL PROBE ASSEMBLY AND METHOD

(71) Applicants: Deli Li, Singapore (SG); Bih Shiou Charles Tsang, Singapore (SG); Wan Sing Ng, Singapore (SG)

(72) Inventors: Deli Li, Singapore (SG); Bih Shiou Charles Tsang, Singapore (SG); Wan Sing Ng, Singapore (SG)

(21) Appl. No.: 13/394,331

(22) PCT Filed: Sep. 6, 2010
(86) PCT No.: PCT/SG10/00323

§ 371 Date: Oct. 22, 2012

(30) Foreign Application Priority Data
Sep. 4, 2009 (SG) 200905941-1

(51) Int. Cl. A61B 8/12

(52) U.S. Cl. 600/466

(57) ABSTRACT

An anorectal probe assembly comprising an anorectal probe, a sonar penetration balloon mounted to said probe, said balloon arranged to receive water to inflate within the rectal cavity a sigmoidscope having a bore into which the probe and balloon have sliding engagement and an adaptor having an anus penetration portion for insertion into the anus and an external portion mounted to the penetration portion wherein the adaptor includes a conduit extending from the penetration portion to the external portion such that on penetration of the anus, the conduit provides fluid communication between the rectal cavity and the external portion, said conduit further connectable to a vacuum source.

### FIELD OF THE INVENTION

The invention refers to the application of ultrasound technology to obtain 3D images for precise diagnoses and surgical procedures. In particular, the invention relates to means of improving the quality and reducing obscurity of said images.

### BACKGROUND

High frequency ultrasound (up to 16 MHz) can penetrate tissue including the rectal wall and obtain detailed information to a depth of a few of millimetres from the tissue surface. However, for a colorectal cancer diagnosis, its application has been limited through poor contact between the ultrasound probe and rectal wall, especially for patients with a tumour, which makes the surface of rectal wall uneven. Further, the presence of entrapped air bubbles within the rectal cavity can obscure otherwise clear images.

### SUMMARY OF INVENTION

In a first aspect, the invention provides an anorectal probe assembly comprising an anorectal probe, a sonar penetration balloon mounted to said probe, said balloon arranged to receive water to inflate within the rectal cavity a sigmoidscope having a bore into which the probe and balloon have sliding engagement and an adaptor having an anus penetration portion for insertion into the anus and an external portion mounted to the penetration portion wherein the adaptor includes a conduit extending from the penetration portion to the external portion such that on penetration of the anus, the conduit provides fluid communication between the rectal cavity and the external portion, said conduit further connectable to a vacuum source.

In a second aspect, the invention provides a method of removing air from a rectal cavity, the method comprising the steps of: inserting an anorectal probe and a sonar penetration balloon into a sigmoidscope; inserting the sigmoidscope into an adaptor, said adaptor having an anus penetration portion, an external portion and a conduit extending from the penetration portion to the external portion; inserting the sigmoidscope into the anus; inserting the anus penetration portion into the anus; connecting the conduit to a vacuum source at the external portion and consequently applying a vacuum to the rectal cavity; withdrawing air trapped between the rectal wall and the balloon through said conduit.

The invention presented here is an anorectal probe assembly for a colorectal examination using ultrasound. The invention provides a better resolution of images by applying a vacuum to the rectal cavity in particular between the rectal wall and the inflated balloon so as to remove trapped air and improve the degree of contact between the balloon and cavity wall. The former solution is directed to reducing obscurity with the latter directed to improving overall image quality.

The external portion may be a collar and the anus penetration portion may be a tube, with both required to be mounted, directly or indirectly, so as to allow the probe and sigmoid scope to pass therethrough.

The external portion or collar may have a sealing ring mounted thereto which provides a circumferential sealing around the sigmoidscope. On insertion of the assembly into the anus, a sealed space is formed with the rectal wall, the adaptor and the sealing engagement between the adaptor and sigmoidscope providing boundary defining this space. On application of the vacuum through the conduit, a negative pressure is established in the rectal cavity, leading to the removal of entrapped air.

To further enhance the seal formed by the adaptor, the external portion may include a plurality of orifices in a contact face, which on insertion of the adaptor comes into proximity with the anus. The orifices may be in fluid communication with the conduit and consequently in communication with the vacuum source. On application of the vacuum source, the orifices apply a negative pressure to the anus and so sealing around the anus to further enhance the sealing effect of the adaptor.

Within the external portion may be positioned a sealing ring valve which may comprise an annulus with holes corresponding to the orifices. The sealing ring may be rotatable such that rotating from a first position may permit the vacuum source to pass through the orifices and a second position which seals the orifices, and so isolating the vacuum source from the orifices, breaking the seal.

### BRIEF DESCRIPTION OF DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIGS. 1A to 1C are various views of an anorectal probe assembly according to one embodiment of the present invention;

FIGS. 2A to 2C are various views of a sonar penetration balloon mounting according to one embodiment of the present invention;

FIGS. 3A to 3C are various views of a coupling according to one embodiment of the present invention;

FIGS. 4A to 4D are various views of an adaptor according to an embodiment of the present invention;

### DETAILED DESCRIPTION

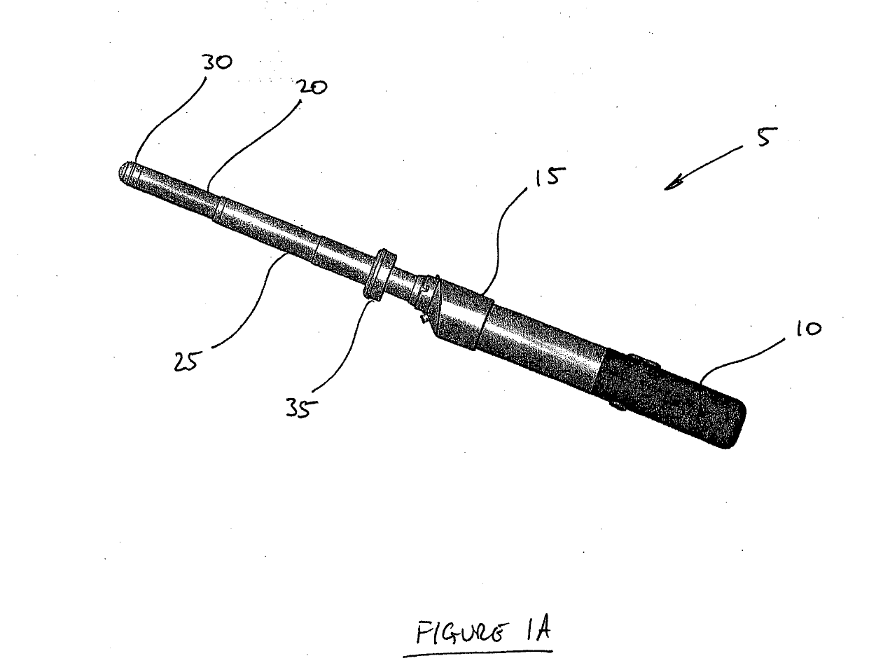

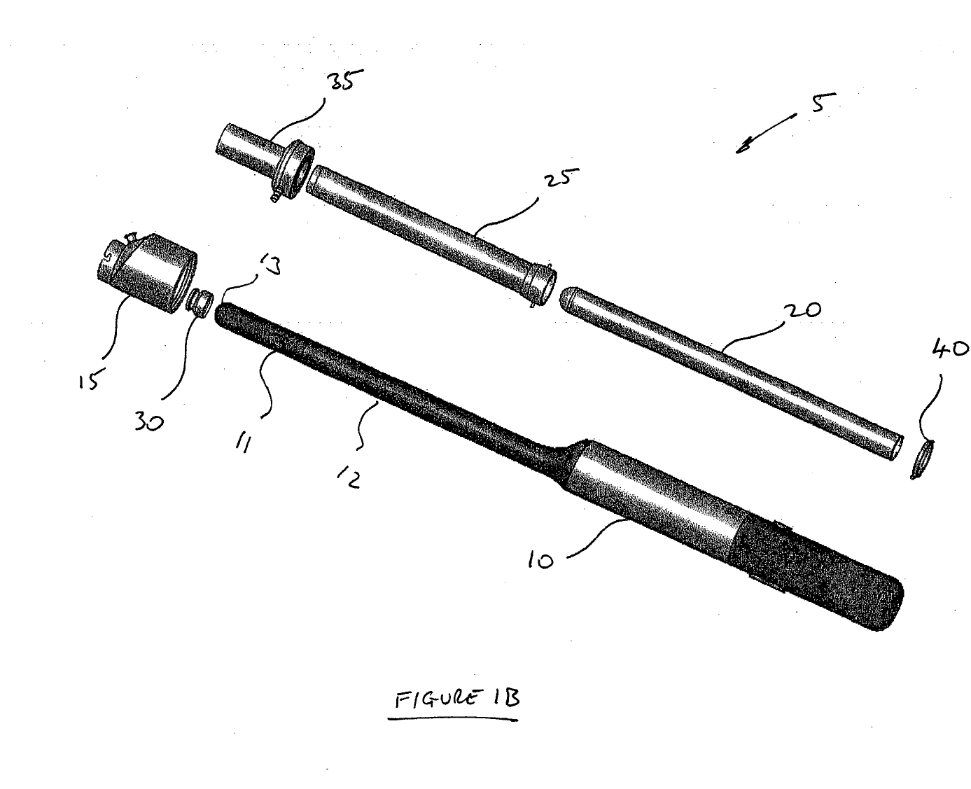

FIGS. 1A, 1B and 1C show an anorectal probe assembly 5 according to one embodiment of the present invention. The assembly 5 comprises an anorectal probe 10 having a shaft 12 for insertion into the rectum and at a distal end of the shaft 12, an ultrasound transducer 11 for creating ultrasound images of the surrounding tissue. An example of such a probe is a BK Medical 2050 Anorectal Transducer.

Mounted to the probe 10 is a balloon 20 which is used to inflate within the rectum by filling with water. Inflating the balloon enlarges the rectum so as to create a clearance between the transducer 11 to improve image quality. The balloon 20 is mounted to the probe through the use of a coupling 15 which fits onto the probe 10 by sliding down the shaft 12 and securing to the probe about an enlargement between the handle and the shaft. The balloon 20 is clamped to the coupling 15 using a quick sealing ring 40 which in this case has a bayonet fitting, that is, a push/twist arrangement to provide a quick clamping effect at the open end of the balloon.

At the distal end 13 of the probe 10 is fitted an end cap 30 which is placed on the probe with the balloon 20 being fixed to the end cap using an O-ring (not shown) or other resilient clamp so as to prevent the balloon inflating longitudinally within the rectum. The end cap 30 therefore acts to restrain the direction of inflation of the balloon and so confine the balloon expansion to the desired area proximate to the transducer 11. Mounted over the balloon 20 is a sigmoidscope 25 and mounted over the top of the sigmoidscope 25 is an adaptor 35.

A procedure according to an embodiment of the present invention may commence with a pre-clinical scan whereupon the end cap 30 is mounted to the probe 10 at the distal end 13. The coupling 15 is then fitted down the shaft 12 and secured to the handle with the probe shaft 12 then inserted into the balloon 20 and clamping one end of the balloon to the coupling 15 using the quick sealing ring 40 and the opposed end of the balloon 20 clamped to the end cap 30 using an O-ring or simple by a nylon string.

To initialise the assembly, the balloon 20 is filled with water and air bubbles expelled from the inflated balloon 20. The balloon is then deflated, removing the water but preventing the inflow of air so as to prevent the balloon having any air bubbles present prior to use.

To commence the clinical scan, the sigmoidscope 25 is inserted in the adaptor 35 with the adaptor 35 connected to a vacuum source such as may be found in an operating theatre or day surgery clinic. The sigmoidscope 25 with the adaptor 35 mounted thereto is inserted into the anus and subsequently to the rectum. The sigmoidscope allows the use of CCD or other devices to do a preliminary visual scan around the rectum.

The probe 10 with the balloon 20 mounted thereto is then inserted through the sigmoidscope into the rectum so as to place the sensor 11 proximate to the area to be investigated by the operator. The adaptor is moved to the anus with the anus penetrating section 85 inserted into the anus so as to bring the external portion or collar 100 proximate to the anus. The vacuum source is engaged with the adaptor through nozzle 105 leading to the orifices 120 engaging the anus in suction and so sealing the anus. At this stage, the rectal cavity is now sealed. With the orifices sealed, the vacuum evacuates the interstitial space between the probe and the rectal wall. The balloon is then inflated with water. With no air bubbles within the rectal cavity to form discontinuities, the balloon can then come in intimate contact with the rectal wall and possibly adopt the shape of any lumps or tumours within the rectum so as to provide a largely uninterrupted layer of the balloon wall against the rectal wall. From this point, the ultrasound scan can begin and images produced to the best available quality.

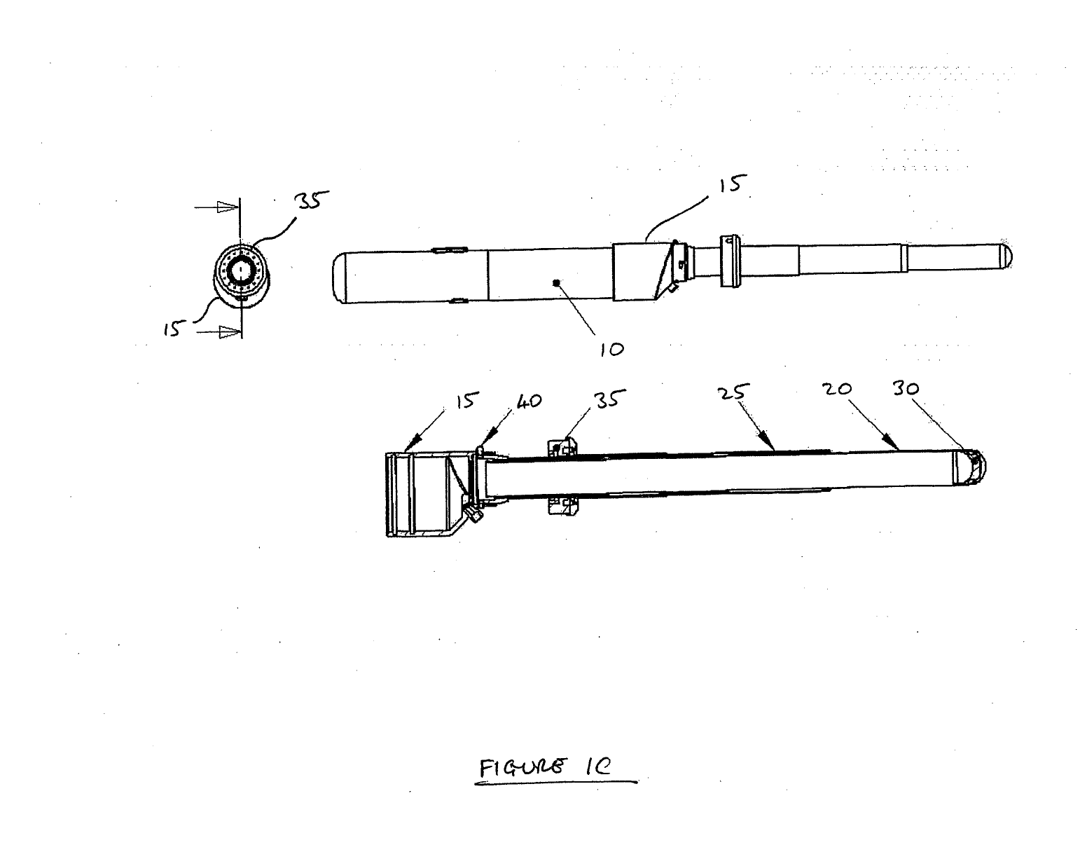

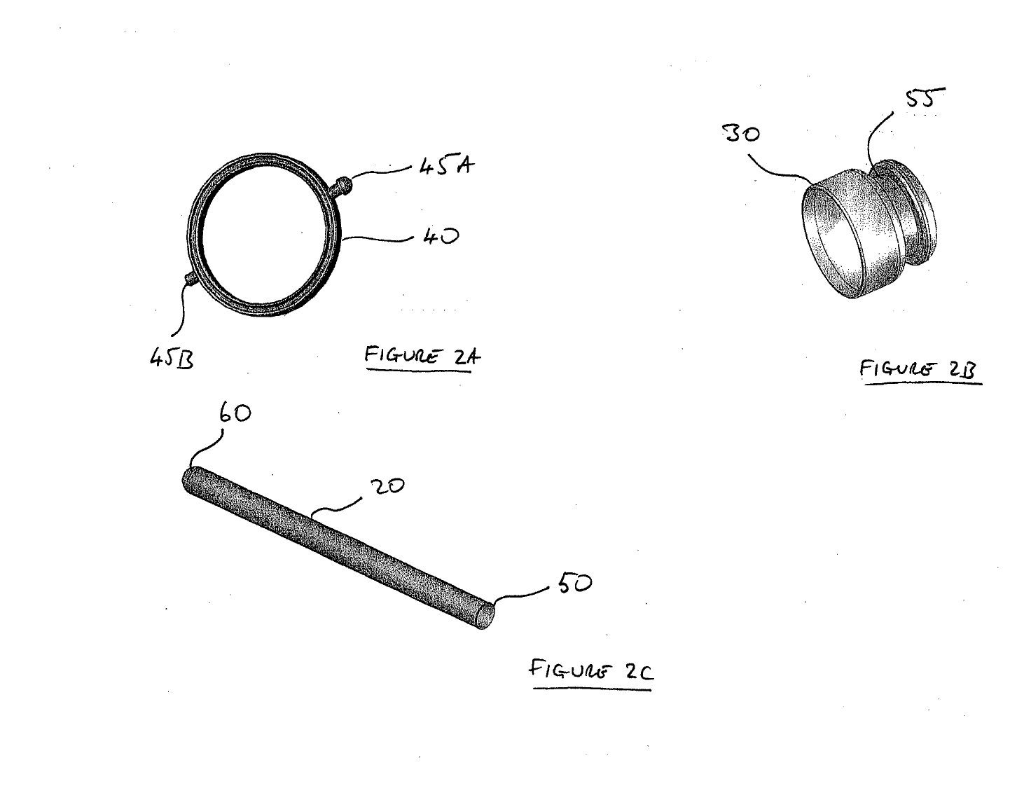

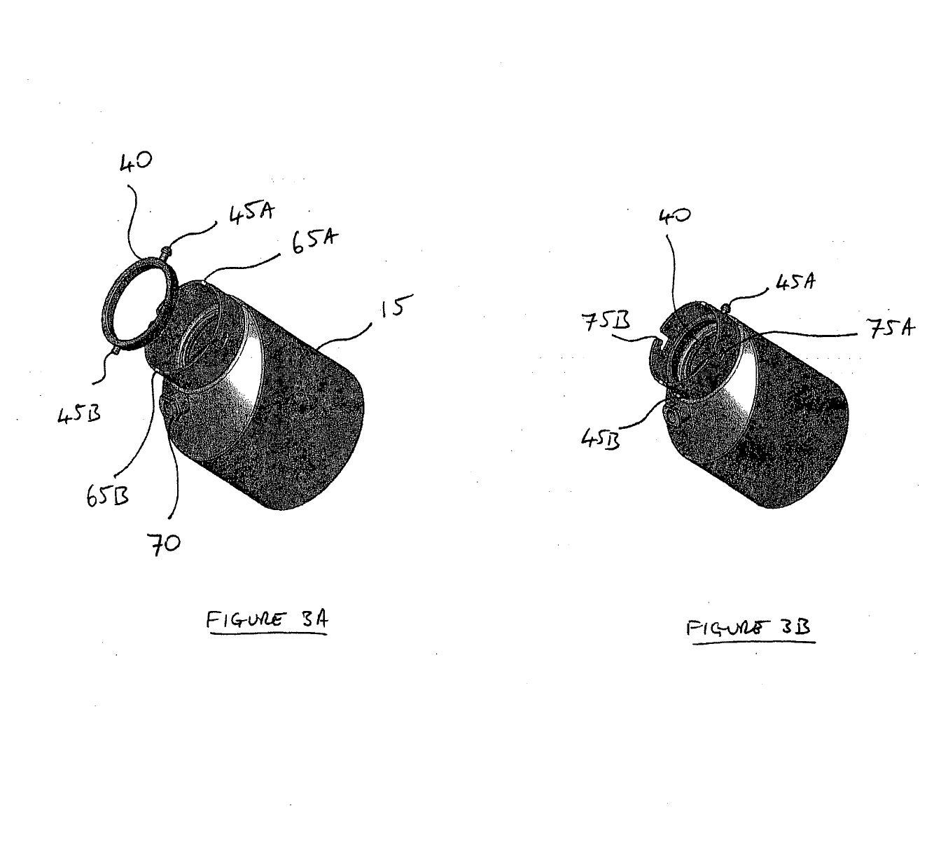

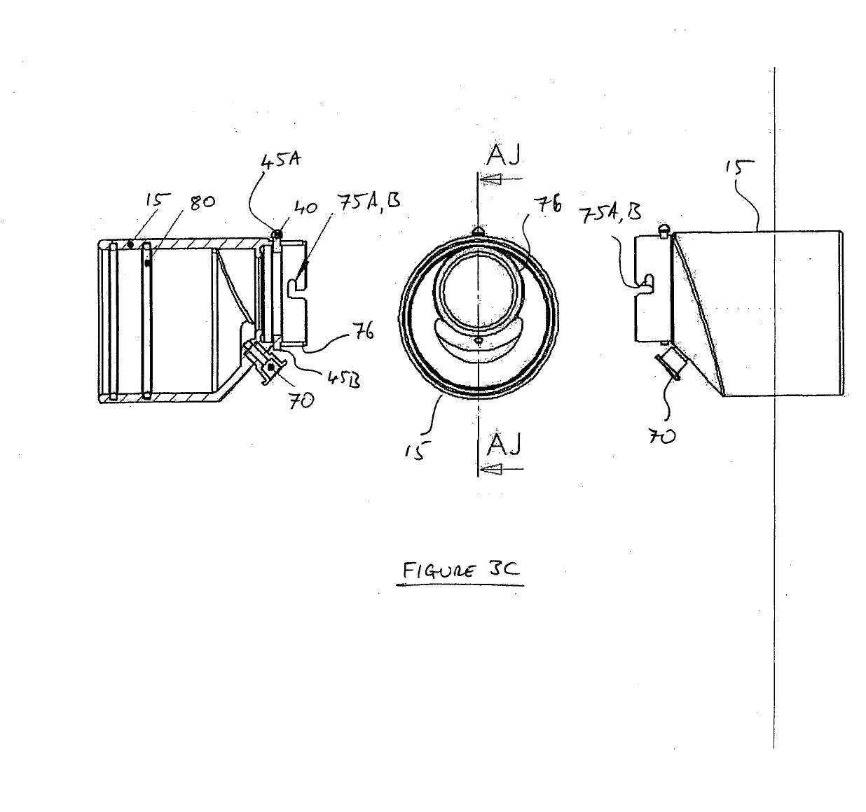

FIGS. 2A to 2C and 3A, 3C provide detailed views of the assembly of the balloon 20 to the coupling 15. FIG. 2A shows the quick sealing ring 40 which is used to clamp the open end 50 of the balloon to the coupling 15. In this embodiment, the quick sealing ring 40 has a bayonet attachment whereby projections 45A, B fit within recesses 75A, B in the coupling. By placing the balloon within the opening 76, the balloon is then compressed and clamped by the insertion of the ring 40 into the bayonet fitting 45A, B, 75A, B and so providing a sealing clamp within the opening 76. This leads to a separation of the inside space of the balloon into 2 chambers, a left chamber towards the balloon stopper and a right chamber towards to the handle of probe. The left chamber is relatively smooth, that is free from ridges and other discontinuities, such that the air could be removed out easily and has no place to hide the air inside. The right chamber could hold some hidden air or leaked air if it happened to avoid the air move to the scanning area.

At the opposed end, the end cap 30 is mounted to the distal end 13 of the probe with the balloon fitting over the top of the shaft 12 of the probe. The end cap 30 may be a metal part adhered to the probe to ensure a permanent or semi-permanent engagement. Alternatively, the end cap 30 may be a plastic part which is selectively removable. In this embodiment, the end cap 30 may be mounted to the probe through a snap fitting to provide a secure connection, but also to be able to detach the end cap 30 after use. Being a plastic part, the snap fit end cap may therefore be disposable, and so avoid issues of cleaning and disinfection for re-use.

Further, the end cap, either for the re-usable or disposable version, may be rounded to provide for a more comfortable insertion into the patient.

Once in place, an O-ring (not shown) fits over the balloon so as to clamp the far end 60 of the balloon 20 into the groove 55 of the end cap 30. Thus the balloon is now fully secured, ready for inflation. Turning to the coupling 15 in particular, the coupling fits over the shaft 12 of the probe and is sealed through a seal 80 within the coupling 15, water inlet 70 can then be connected to a water source so as to flow water through the opening 76 so as to inflate the balloon.

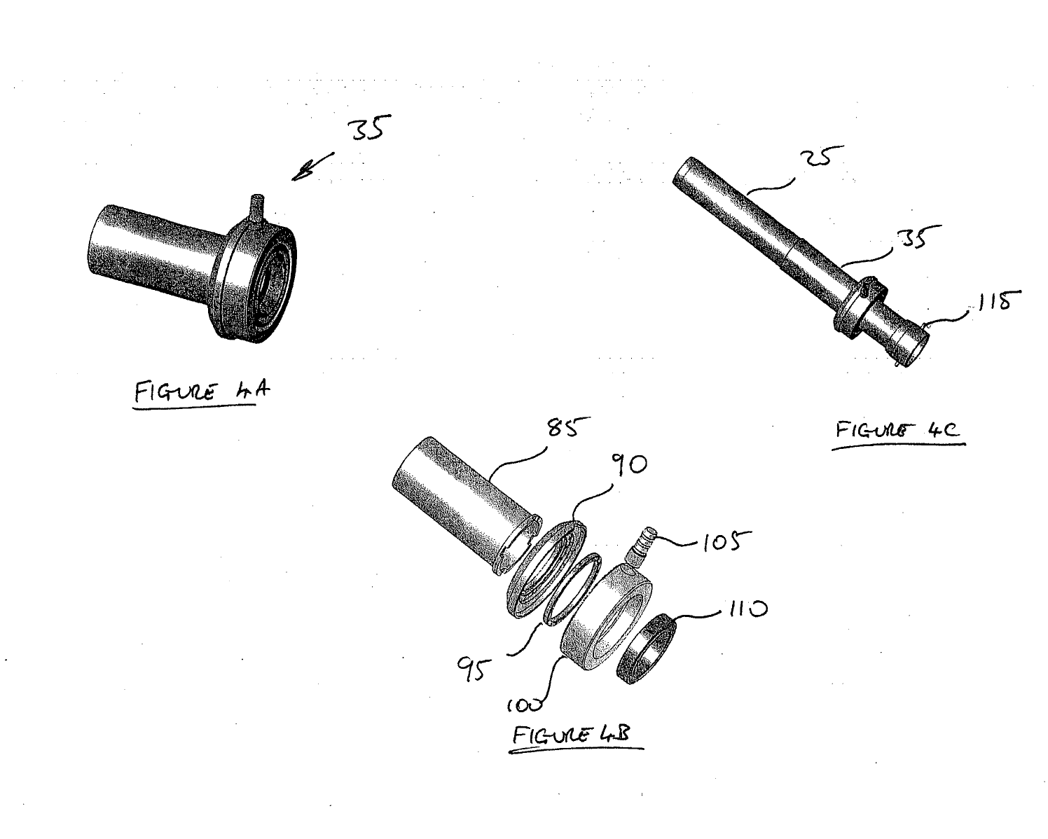

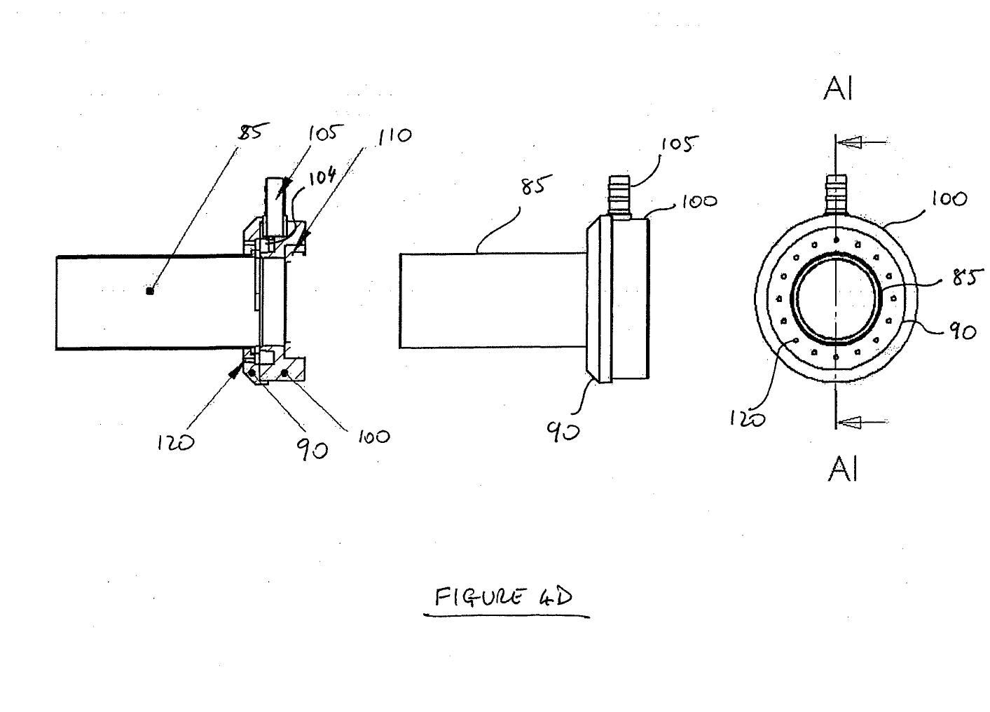

FIGS. 4A to 4D show the adaptor 35 and its connection with the sigmoidscope 25. The adaptor itself comprises an assembly which may be of a unitary construction or an assembly having an anus penetration section 85 in the form of a tube mounted to a front ring 90 and subsequently mounted to an external portion 100 in the form of a collar. Mounted between the collar 100 and the front ring 90 is a vacuum ring valve 95 which is rotatable within the adaptor 35.

Within the front ring 90 is placed an array of orifices 120 which are in communication with a conduit 104. The conduit 104 extends from the anus penetration portion 85 to the external portion 100 which in turn is connected to a nozzle 105. The nozzle 105 is connectable to a vacuum source and thus through the conduit 104, the anus penetration portion 85 can provide vacuum communication between the rectal cavity and the vacuum source on insertion. With the orifices 120 also in communication with the conduit 104, the orifices 120 also provide a vacuum seal against the anus when the adaptor is brought proximate to the anus and so providing a seal for the rectal cavity. Once the anus is sealed the vacuum is provided through the conduit 104 so as to remove any air between the probe and the rectal wall prior to inflation of the balloon 20.

To ensure a complete seal between the sigmoidscope 25 and the adaptor 35, the collar 100 includes a sealing ring 110 which permits a sliding engagement between the sigmoidscope and adaptor but when stationary provides sufficient circumferential contact so as to seal against any loss of negative pressure created by the vacuum within the rectal cavity.

1. An anorectal probe assembly comprising
an anorectal probe, a sonar penetration balloon mounted to said probe, said balloon arranged to receive water to inflate within the rectal cavity a sigmoidscope having a bore into which the probe and balloon have sliding engagement and
an adaptor having an anus penetration portion for insertion into the anus and an external portion mounted to the penetration portion wherein the adaptor includes a conduit extending from the penetration portion to the external portion such that on penetration of the anus, the conduit provides fluid communication between the rectal cavity and the external portion, said conduit further connectable to a vacuum source.

2. The anorectal probe assembly according to claim 1 further including a coupling to mount to the probe so as to fix and seal an open end of said balloon to the probe.

3. The anorectal probe assembly according to claim 2 wherein the coupling includes a water inlet for connection to a water source for inflating the balloon with water.

4. The anorectal probe assembly according to claim 2 wherein the coupling includes a sealing ring to lock into a recess of the coupling whilst clamping and sealing the balloon.

5. The anorectal probe assembly according to claim 2 wherein the coupling includes a recess for engaging the sigmoidscope.

6. The anorectal probe assembly according to claim 1 further including an end cap for mounting to a distal end of the probe, said end cap arranged to engage a distal end of said balloon so as to prevent longitudinal inflation of the balloon passed the distal end of said probe.

7. The anorectal probe assembly according to claim 1 wherein the external portion is a collar having an air nozzle fixed thereto said nozzle arranged to connect to a vacuum source.

8. The anorectal probe assembly according to claim 1 wherein the adaptor includes a sealing ring mounted to the external portion such that the sigmoidscope is placed through said sealing ring so as to provide a circumferential seal around said sigmoidscope.

9. The anorectal probe assembly according to claim 1 wherein the penetration portion includes a tube mounted to the external portion.

10. The anorectal probe assembly according to claim 1 wherein the external portion includes a plurality of orifices on a contact face of said external portion, said orifices in fluid communication with the conduit such that on application of the vacuum source a negative pressure is applied through said orifices.

11. The anorectal probe assembly according to claim 1 wherein the adaptor includes a sealing valve ring positioned within the external portion, said sealing valve ring having holes placed around said ring, said ring mounted so as to be rotatable from a first position where upon the holes correspond to the orifices and a second position whereupon the orifices are sealed from the vacuum source.

12. A method of removing air from a rectal cavity, the method comprising:
inserting an anorectal probe and a sonar penetration balloon into a sigmoidscope;
inserting the sigmoidscope into an adaptor, said adaptor having an anus penetration portion, an external portion and a conduit extending from the penetration portion to the external portion.
inserting the sigmoidscope into the anus;
inserting the anus penetration portion into the anus;
connecting the conduit to a vacuum source at the external portion and consequently applying a vacuum to the rectal cavity;
withdrawing air trapped between the rectal wall and the balloon through said conduit.
